(19) United States
(12) Patent Application Publication
(10) Pub. No.: US 2004/0259770 A1
(43) Pub. Date: Dec. 23, 2004

(54) Method for determining leukocyte activation

(75) Inventor: Larry L. Thomas, Chicago, IL (US)

Correspondence Address: FOLEY & LARDNER, 150 EAST GILMAN STREET, P.O. BOX 1497, MADISON, WI, 53701-1497, US

(73) Assignee: Rush University Medical Center

(21) Appl. No.: 10/869,154

(22) Filed: Jun. 16, 2004

Related U.S. Application Data
(60) Provisional application No. 60/479,634, filed on Jun. 19, 2003.

(51) Int. Cl. A61K 38/40; C12Q 1/00

(52) U.S. Cl. 514/6; 435/4

(57) ABSTRACT

This invention relates to assays for determining whether lactoferrin-mediated leukocyte activation occurs and measuring the strength of any reaction. Generally, lactoferrin, in soluble or immobilized form, is contacted with a cell population containing leukocytes and the level of leukocyte activation is determined typically through superoxide production. The assays can also be carried out in the presence of one or more potential regulators of lactoferrin-mediated leukocyte activation to determine any effect on activation.

This application claims priority to U.S. Provisional Patent Application No. 60/479,634, filed Jun. 19, 2003, the entire contents of which are hereby incorporated.

### STATEMENT REGARDING FEDERALLY SPONSORED RESEARCH

This invention was made with United States Government support in the form of a grant from the National Institutes of Health, Grant No. R21 AI 48160. The United States Government has certain rights in this invention.

### FIELD OF THE INVENTION

The present invention relates to methods for detecting leukocyte activation by lactoferrin. More particularly, the invention relates to the stimulation of eosinophil superoxide production, leukotriene C4 production and degranulation as a result of an interaction with immobilized lactoferrin.

### BACKGROUND

The environment contains a variety of infectious microbial agents, such as viruses, bacteria, fungi and parasites, any one of which can cause pathological damage to the host organism. Consequently, most organisms, such as mammals, i.e. humans, have developed an immune system, a regulatory system that maintains homeostasis by protecting the body against not only foreign particles, such as pathogenic microbial agents, but also native cells that have undergone neoplastic transformation. The immune system exerts its control within the body by virtue of circulating components, humoral and cellular, capable of acting at sites removed from their point of origin. The complexity of the immune system is derived from an intricate communications network capable of exerting multiple effects based on relatively distinct cell types, the most important of which are leukocytes, Leukocytes are categorized into neutrophils, eosinophils, monocytes, macrophages, and lymphocytes.

Inflammation is the body's response to invasion or an injury, such as an invasion by an infectious microbial agent and includes three broad actions. First, the blood supply is increased to the area. Second, capillary permeability is increased, thereby permitting larger molecules to reach the site of infection. Third, leukocytes, migrate out of the capillaries and into the surrounding tissue. Once in the tissue, the leukocytes migrate to the site of infection or injury by chemotaxis. At the site of infection, leukocytes perform phagocytic and degradative functions to combat the infectious agent. As part of their immune response, some leukocytes generate superoxide anions, reactive oxygen species to kill infectious material and adhere to epithelial cells of mucosal surfaces or vascular endothelial cells of the blood vessels. These events manifest themselves as inflammation. As a consequence, the host can experience undesirable side effects during the elimination of the infectious agent such as, pain, swelling about the site, and nausea. Examples of conditions which cause these reactions to occur include clamping or tourniquet vessel-induced ischemia reperfusion injury, chronic inflammatory conditions such as asthma, rheumatoid arthritis, and inflammatory bowel disease, as well as autoimmune diseases.

Additionally, aberrant activation of phagocytic cells leads to the generation of superoxide anion which, when released to the extracellular milieu, can evoke damage to surrounding tissues. Reactive oxygen species derived from leukocyte oxygen burst can play a deleterious role in generating secondary products that lead to loss of function. The leukocyte-derived oxygen radicals and other toxic products that are normally intended for killing of microbial agents once they spill over into the surrounding tissue can lead to second organ injury, most notably in the lung and cardiac tissues.

One condition where this is most apparent is the complex disorder asthma. Both hereditary and environmental factors, including allergies, viral infections, and irritants are involved in the onset of asthma and its inflammatory exacerbations. Even patients with mild disease show airway inflammation, including infiltration of the mucosa and epithelium with activated T cells, mast cells, and eosinophils. T cells and mast cells release cytokines that promote eosinophil growth and maturation and the production of IgE antibodies, and these, in turn, increase microvascular permeability, disrupt the epithelium, and stimulate neural reflexes and mucus-secreting glands. The result is airway hyperreactivity, bronchoconstriction, and hypersecretion, manifested by wheezing, coughing, and dyspnea. Accordingly, there is a continuing need to understand the underlying mechanisms that prompt leukocyte activation and develop assays that measure leukocyte responses to immune stimuli.

### BRIEF DESCRIPTION OF THE DRAWINGS

The invention will be more readily understood by reference to the following description, taken with the accompanying drawings, in which:

FIG. 1 depicts the stimulation of eosinophil superoxide (O₂⁻) production by immobilized lactoferrin and immobilized secretory IgA. Eosinophils were incubated in tissue culture wells preincubated with the indicated concentrations of lactoferrin or secretory IgA as described elsewhere herein. Representative time courses from a single assay are shown for superoxide production stimulated by the indicated concentrations of immobilized lactoferrin (A) and immobilized secretory IgA (B). The concentration requirements for stimulation of superoxide production by immobilized lactoferrin (C) and immobilized secretory IgA (D) determined at the 2-hour time point are shown as the mean±SEM for five assays, including the assay shown in A and B. The values are corrected for spontaneous superoxide production (0.7±0.6 nanomoles of superoxide per 10⁵ eosinophils) and, in C, for GM-CSF stimulated superoxide production (2.8±0.7 nanomoles of superoxide per 10⁵ eosinophils) (*p<0.05 compared to the spontaneous value);

FIG. 2 depicts the stimulation of eosinophil superoxide (O₂⁻) production by immobilized lactoferrin and immobilized transferrin. Eosinophils were incubated with the indicated concentrations of immobilized lactoferrin or immobilized transferrin as described for FIG. 1. Results are the mean±SEM for three assays after subtraction of the spontaneous value (0.3±0.2 nanomoles per 10⁵ eosinophils);

FIG. 3 depicts a comparison of neutrophil and eosinophil superoxide (O₂⁻) production in response to immobilized lactoferrin and immobilized secretory IgA. Neutrophils (A) and eosinophils (B) isolated from the same individuals were incubated with the indicated concentrations of immobilized lactoferrin or secretory IgA as described for FIG. 1. Results are the mean±SEM for four assays after subtraction of the spontaneous values, which were <0.1 nanomoles per 10⁵ cells for both neutrophils and eosinophils (*p<0.05 compared to the value for immobilized secretory IgA);

FIG. 4 depicts the flow cytometric analysis of binding of lactoferrin by eosinophils. Eosinophils were incubated in the absence (A) or presence (B) of 30 micrograms per milliliter of lactoferrin for 90 minutes at 40° C. as described elsewhere herein. Bound lactoferrin was detected by flow cytometry after subsequently incubating the cells with 1.5 micrograms of FITC-IgG anti-human lactoferrin (heavy line) or with FITC-IgG (fine line) as described elsewhere herein. Similar results were obtained in two additional assays;

FIG. 5 depicts the binding of ¹²⁵I-labeled-lactoferrin by eosinophils. Eosinophils were incubated with the indicated concentrations of ¹²⁵I-labeled-lactoferrin (Lf) (34,800 cpm/pmole) for 2 hours at room temperature as described. Specific binding is shown as the mean±SEM of three to six experiments using eosinophils from five individuals. Specific binding was obtained by subtracting non-specific binding measured in the presence of 5 micromolar unlabeled lactoferrin from total binding;

FIG. 6 depicts the effect of soluble lactoferrin on eosinophil superoxide (O₂⁻) production. Eosinophils were incubated with the indicated concentrations of soluble or immobilized lactoferrin (A) as described for FIG. 1. Results are the mean±SEM for four assays after subtraction of spontaneous production (<0.1 nanomoles per 10⁵ eosinophils) (*p<0.05 compared to the value for immobilized lactoferrin). Eosinophils were incubated with 30 micrograms per milliliter of immobilized lactoferrin (B) in the presence of the indicated concentrations of soluble lactoferrin. Results are the mean±SEM for three assays after subtraction of the spontaneous value (0.2±0.1 nanomoles per 10⁵ eosinophils);

FIG. 7 depicts the stimulation of eosinophil EDN release and leukotriene C4 release by immobilized lactoferrin. Eosinophils were incubated for 4 hours (A) or 1 hour (B) at 37° C. in tissue culture wells preincubated with the indicated concentrations of lactoferrin in the absence and presence of 100 picograms per milliliter of GM-CSF. EDN release (A) and leukotriene C4 release (B) are shown as the mean±SEM for five assays and four assays, respectively. (*p<0.05 compared to the additive effects of immobilized lactoferrin and GM-CSF);

FIG. 8 depicts the stimulation of eosinophil superoxide (O₂⁻) production by immobilized deglycosylated lactoferrin (Lf). A. Eosinophils were incubated for 120 minutes at 37° C. in tissue culture wells preincubated with the indicated concentrations of deglycosylated lactoferrin (PNGase F-treated) or mock-deglycosylated lactoferrin (subjected to the same treatment as deglycosylated lactoferrin but in the absence of PNGase F). Results are the mean±SEM for four assays after subtraction of the spontaneous value (0.2±0.2 nanomoles per 10⁵ eosinophils). B. Coomassie-stained PAGE gel of 10 micrograms (lane 1) control, (lane 2) mock-deglycosylated, and (lane 3) deglycosylated lactoferrin. C. Western blot of 0.2 micrograms (lane 1) control, (lane 2) mock-deglycosylated, and (lane 3) deglycosylated lactoferrin detected with HRP-conjugated ConA; and

FIG. 9 depicts the effect of heparin and chondroitin sulfate on eosinophil superoxide (O₂⁻) production stimulated by immobilized lactoferrin (LI). Eosinophils were incubated in the absence or presence of the indicated concentrations of heparin or chondroitin sulfate for 120 minutes at 37° C. in tissue culture wells preincubated with 30 micrograms per milliliter of lactoferrin. Results are the mean±SEM for four assays after subtraction of the spontaneous value (0.6±0.4 nanomoles per 10⁵ eosinophils) (*p<0.05 compared to control).

### SUMMARY OF THE INVENTION

One aspect of the present invention provides a method for assaying leukocyte activation, including the steps of (a) contacting one or more leukocytes with lactoferrin, a portion of lactoferrin or a derivative of lactoferrin or a portion thereof; and (b) determining whether the one or more leukocytes are activated by the contact with the lactoferrin, the portion of lactoferrin or the derivative of lactoferrin or a portion thereof. In some of the methods, the lactoferrin, the portion of lactoferrin or the derivative of lactoferrin or a portion thereof are immobilized, such as on a surface of a piece of disposable lab equipment. In these or other methods the leukocytes can include eosinophils, neutrophils and combinations thereof. In still other aspects of the invention step (b) can further include quantifying the level of activation of the one or more leukocytes. Detection in step (b) can include one or more of: (i) detecting superoxide production by the one or more leukocytes, (ii) detecting eosinophil-derived neurotoxin (EDN) release by the one or more leukocytes; (iii) detecting degranulation of the one or more leukocytes; (iv) detecting production of one or more leukotrienes; (v) detecting whether the lactoferrin, the portion of lactoferrin or derivative of lactoferrin or a portion thereof binds to the leukocyte; (vi) detecting production of one or more cytokines; and (vii) combinations of (i)-(vi). Other methods further involve immobilizing the lactoferrin, the portion of lactoferrin or derivative of lactoferrin or a portion thereof on a surface.

Any of the present methods can be carried out in the presence of one or more potential modulators, such as inhibitors or stimulators, of leukocyte activation. In some of these methods, a control can be run, such that the control is performed in the absence of the one or more potential inhibitors of leukocyte activation. The leukocyte activation assay in the presence of the one or more potential inhibitors can then be compared to the leukocyte activation assay in the absence of the one or more potential inhibitors.

The present invention also provides kits for carrying out the disclosed methods that include (a) instructions for carrying out any of the methods described herein; and (b) one or more reagents for performing the described methods.

### DETAILED DESCRIPTION OF THE INVENTION

This application relates to U.S. Provisional Patent Application No. 60/335,241, filed Oct. 30, 2001, 60/384,200, filed May 30, 2002, 60/388,796, filed Jun. 13, 2002, and 60/389,045, filed Jun. 14, 2002, the entire contents of all of which are hereby incorporated by reference. The present invention provides techniques for determining whether lactoferrin mediated activation occurs in a cell population including leukocytes. These techniques exploit the finding that lactoferrin in soluble and immobilized form activates different subsets of leukocytes to varying degrees. These results are useful for at least understanding the immune response mechanisms leukocytes use to defend the body against infection, parasitic invasion and inflammatory stimuli. The present techniques also provide methods for determining whether a particular compound or agent regulates leukocytic immune responses and the compound or agent's efficacy in regulating the immune response.

According to these methods, one or more leukocytes, which can be present in a cell population made up primarily of leukocytes or can be leukocytes in a mixed cell population, are contacted with lactoferrin, fragments of lactoferrin, derivatives of lactoferrin, or the like, including combinations of all of the forgoing (collectively referred to as lactoferrin for ease of discussion). Generally, contacting involves placing the leukocytes and lactoferrin together into a culture dish or multi-well plate. The effect of the lactoferrin on activation, or lack thereof, of the leukocyte is determined, and can be measured if desired. The present methods preferably use cell populations that are primarily composed of leukocytes. More preferably the leukocytes will make up greater than 90 percent, such as 95, 99 percent or more, of the cells in the population. One disclosed method for obtaining a cell sample enriched in leukocytes is disclosed in U.S. Pat. No. 5,785,869. In some preferred embodiments the leukocytes are isolated from a cell population obtained from a patient or patients having a leukocyte population of interest, such as in an individual suffering from a leukocyte-related disorder or condition such as asthma, or an individual whose leukocytes display a genetic disorder. Accordingly, the leukocyte sample from the patient or patients of interest can be used to test the sensitivity of those leukocytes to lactoferrin-mediated activation against a control sample from an individual not having the disorder. Such comparisons can suggest a potential course of treatment for the patient.

Preferably, the lactoferrin is immobilized on a solid support, such as in a culture dish, multi-well plate or on beads, such as polymer or metal (e.g. iron) beads, however the lactoferrin can be unbound in solution if desired. However, less activation of the leukocytes is readily apparent using unbound lactoferrin. As the methods preferably utilize immobilized lactoferrin, the methods can also include the active step of immobilizing the lactoferrin on a solid support, although the present methods can also take advantage of a solid support on which lactoferrin has been immobilized separately from performing the method. In preferred embodiments of the present methods, lactoferrin is present in amounts typically found in the in airway surface liquid of the lungs and in the effective concentration range for stimulation of eosinophil superoxide production by immobilized secretory IgA. These amounts are generally from about 1 microgram per milliliter to about 100 micrograms per milliliter. More preferably, lactoferrin concentrations range from about 20 to 50 micrograms per milliliter, or 30 to 40 micrograms per milliliter. In some embodiments, particularly where lower concentrations of lactoferrin are used, granulocyte macrophage—colony stimulating factor (GM-CSF) in an amount up to about 50, 100, 250, 500 or 1000 picograms per milliliter can be added as a potential stimulator of the lactoferrin-mediated leukocyte activation. Higher concentrations of lactoferrin, including up to about 250, 500 or 1000 micrograms per milliliter can be used in some methods, particularly if inhibition of the lactoferrin mechanism is suspected.

Leukocyte reaction or activation to or by the presence of lactoferrin can be determined by a number of indicators including, but not limited to, superoxide production, eicosanoid production, degranulation, sensitivity to signaling molecules such as GM-CSF; eosinophil-derived neurotoxin (EDN) release, leukotriene production, lactoferrin binding, cytokine production, and combinations of the aforementioned. Activation of the leukocyte can further be determined based on a control assay where the leukocytes are treated in the same manner as above, except that leukocytes are not exposed to the lactoferrin. Non-specific protein binding sites on the leukocyte can be blocked in the present methods as desired, such as by treating the leukocytes with human serum albumin (HSA) or the like.

Leukocytes (white blood cells) come in two classes based on nuclear morphology: polymorphonuclear granulocytes that have segmented nuclei and cell-specific cytoplasmic granules; and mononuclear agranulocytes that have nonsegmented nuclei and no specific cytoplasmic granules. Polymorphonuclear granulocytes include basophils, eosinophils and neutrophils (which are the most common). Mononuclear agranulocytes include monocytes and lymphoctes. In some of the present methods it is preferable to use cell populations that are made up almost entirely of only one of the five mentioned (basophil, eosinophil, neutrophil, monocyte and lymphocyte) leukocyte subtypes. In these methods the chosen leukocyte subtype will generally be greater than 90, 95 or 99 percent of the leukocyte cells in the sample. The present methods can also provide comparative assays using the same conditions and steps, except where different leukocyte subtypes are used in the separate assays. These comparative assays can determine the effect of lactoferrin on the specific cell subtype tested in relation to the other subtypes. Such comparative data can help pinpoint specific cell subtypes that can be tested or targeted for regulation. In this embodiment, as in others, it may be desirable to utilize leukocytes that are known to have a certain defect, such as a genetic defect. Any desired leukocytes can be used in the present methods although neutrophils and eosinophils are preferred.

Leukocytes have been found to be directly involved in a wide array of immune responses, and disorders. In certain inflammatory diseases, infiltration of leukocytes into sites of inflammation is observed. For example, eosinophil infiltration of the bronchus in asthma (Ohkawara, Y. et al., Am. J. Respir. Cell Mol. Biol., 12 4-12 (1995)); infiltration of T lymphocytes and eosinophils into the skin in atopic dermatitis (Wakita, H. et al., J. Cutan. Pathol., 21 33-39 (1994)) or contact dermatitis (Satoh, T. et al., Eur. J. Immunol., 27, 85-91 (1997)); and infiltration of various leukocytes into rheumatoid synovial tissue (Tak, P P. et al., Clin. Immunol. Immunopathol., 77, 236-242 (1995)), have been reported.

Eosinophils are a polymorphonuclear leukocyte, typically containing strongly staining secondary granules. Eosinophils typically make up 2-5% of the leukocytes in a healthy human. They are important effector cells in host defense especially against helminth or other parasite infection. Eosinophil levels are elevated during parasitic infection and during allergic reactions, especially type I hypersensitivity responses. Elevated numbers of eosinophils in the blood (eosinophilia) can contribute to the pathogenesis of a variety of inflammatory disorders, most notably allergic diseases such as asthma (Wardlaw, A. J., et al., Adv. Immunol. 60:151 (1995); Rothenberg, M. E., New Engl. J. Med. 338:1592 (1998)). In particular, the local accumulation of eosinophils within tissues such as the lungs is a hallmark of allergic disorders, and the numerous pro-inflammatory mediators released by eosinophils are strongly implicated in the pathophysiological changes in asthma and other allergic inflammatory diseases (Gleich, G. J., J. Allergy Clin. Immunol. 105:651(2000)). Eosinophils are strongly implicated in the pathogenesis in asthma, particularly in the damage to the airway epithelial lining. Accordingly, elucidation of the mechanisms responsible for eosinophil recruitment and activation is critical to the full understanding of eosinophil-associated disorders.

Eosinophils are postulated to contribute to the pathogenesis in asthma and other allergic diseases through the release of their granule contents and production of reactive oxygen intermediates (Gleich, G. J., J. Allergy Clin. Immunol. 105:651 (2000); Wu, W., et al., J. Clin. Invest. 105:1455 (2000)). The capacity of immobilized lactoferrin to stimulate eosinophil superoxide production and degranulation suggests that lactoferrin adherent to the surface epithelium may constitute one mechanism for initiating these events within the airway. Moreover, the present results, along with the activity of immobilized secretory IgA (Abu-Ghazaleh, R. I., et al., J. Immunol. 142:2393 (1989); Motegi, Y., and H. Kita, J. Immunol. 161:4340 (1998)) and the recent finding that Clara cell secretory 10-kDa protein can limit eosinophil-associated lung inflammation (Chen, L. C., et al., J. Immunol. 167:3025 (2001)), indicate that prominent constituents within the airway surface liquid may contribute to the regulation of eosinophil activation within the airway. Immobilized secretory IgA is one of the most potent stimuli for eosinophil superoxide production and degranulation (Abu-Ghazaleh, R. I., et al., J. Immunol. 142:2393 (1989)). The increased potency of immobilized secretory IgA relative to either immobilized IgG or immobilized serum IgA (Abu-Ghazaleh, R. I., et al., J. Immunol. 142:2393 (1989)) reflects the capacity of immobilized secretory component to also stimulate eosinophil superoxide production and degranulation (Motegi, Y., and H. Kita, J. Immunol. 161:4340(1998)). Although concomitant neutrophil infiltration and activation within the lungs could constitute an additional source of lactoferrin for eosinophil activation, it is worth noting that oxidizing pollutants have been reported to increase lactoferrin synthesis by bronchial epithelial glands (Ghio, A. J., et al., Am. J. Physiol. 274:L728 (1998)). Further, the finding that eosinophil cationic protein stimulates lactoferrin release by serous glands in explants of human nasal mucosa (Roca-Ferrer, et al., J. Allergy Clin. Immunol. 108:87 (2001)) raises the possibility that eosinophil activation by immobilized lactoferrin may provide feedback reinforcement for additional degranulation and oxidant production by the eosinophils.

A hallmark of eosinophil-mediated inflammation in the lungs is damage of the airway epithelial lining (Gleich, G. J., J. Allergy Clin. Immunol. 105:651 (2000)). The damage to airway epithelium is attributed to the cytotoxic actions of eosinophil granule proteins such as major basic protein and to oxidants produced by the interaction of eosinophil peroxidase and hydrogen peroxide in the presence of bromine (Wardlaw, A. J., et al., Adv. Immunol. 60:151 (1995); Gleich, G. J., J. Allergy Clin. Immunol. 105:651 (2000); Wu, W., et al., J. Clin. Invest. 105:1455 (2000)). Secretory IgA is the prominent antibody class in mucosal secretions and is one of the most effective stimuli for eosinophil superoxide production and degranulation when immobilized on a non-phagocytosable surface (Abu-Ghazaleh, R. I., et al., J. Immunol. 142:2393 (1989); Motegi, Y., and H. Kita, J. Immunol. 161:4340 (1998)). This finding has demonstrated the potential for eosinophil activation to occur within the airway, a conclusion supported by the presence of eosinophil granule proteins in mucous plugs and along the mucosal epithelial surface in asthmatic airways (Filley, W. V., et al., Lancet 2:11 (1982)).

Lactoferrin is a 78-80 kilodalton (kDa) multifunctional glycoprotein distributed in external secretions that bathe the body surfaces and in the secondary granules of certain leukocytes (Lonnerdal, B., and S. Iyer, Annu. Rev. Nutr. 15:93 (1995); Borregaard, N., and J. B. Cowland, Blood 89:3503 (1997)). The distribution in the secondary granules results from the synthesis of lactoferrin by glandular epithelial cells and mature neutrophils. Although frequently used as a marker for neutrophil degranulation at sites of inflammation, lactoferrin is also one of the more abundant proteins in the airway surface liquid covering the mucosal epithelium (Travis, S. M., et al., Am. J. Respir. Cell. Mol. Biol. 20:872 (1999)). The three-dimensional structure of lactoferrin and the related molecule transferrin have been precisely defined by X-ray crystallographic analysis (Anderson et al., J. Mol. Biol. 209: 711-734 (1989); Lindley et al., Biochem. 27: 5804-5812(1988)). Lactoferrin is folded into two globular lobes, corresponding roughly to the amino- and carboxy-terminal halves of the protein. Each lobe can reversibly bind iron with high affinity (Aisen et al., Ann. Rev. Biochem. 49: 357-393 (1980). Given its localization and its bacteristatic and bactericidal properties (Reiter, B., et al., Immunology 28:83 (1975); Arnold, R. R., et al., Science 197:263 (1977); Travis, S. M., et al., Curr. Opin. Immunol. 13:89 (2001)), lactoferrin is postulated to contribute to the bacterial host defense function of neutrophils and to play a protective role against bacterial pathogens contacting the airway mucosa (Travis, S. M., et al., Curr. Opin. Immunol. 13:89 (2001)). It now appears that the biological actions of lactoferrin are not restricted to its bacteristatic and bactericidal properties. A wide array of actions have been reported for lactoferrin (Lonnerdal, B., and S. Iyer, Annu. Rev. Nutr. 15:93 (1995); Vorland, L. H., Apmis 107:971 (1999); Baveye, S., E. et al., Clin. Chem. Lab. Med. 37:281 (1999)): including cellular growth promotion; regulation of myelopoiesis; immunomodulatory properties, including stimulating neutrophil aggregation and adhesion (Oseas, R., H., et al., Blood 57:939 (1981); Kurose, I., et al., J. Leukoc. Biol. 55:771 (1994)) and enhancing NK cell activity (Damiens, E., et al., Biochim. Biophys. Acta 1402:277 (1998)).

The lactoferrin suitable for use in the present invention is not particularly limited. Naturally occurring lactoferrin is suitable for use in the present methods as are variants of lactoferrin, fragments of lactoferrin and products of lactoferrin resulting from enzymatic treatment and digestion, such as deglycosylated lactoferrin. Lactoferrins from various species, such as murine, porcine, bovine, equine, homo sapiens, or the like, can also be used. Fragments and variants of lactoferrin are disclosed in U.S. Pat. Nos. 6,111,081; 6,333,311 and 5,304,633. Lactoferrin used in the present methods and kits can be either isolated from a naturally occurring source, such as disclosed in U.S. Pat. Nos. 5,919,913; 5,861,491; 5,849,885; 5,596,082; 5,516,675; 5,149,647; 4,997,914; 4,791,193; 4,668,771; and 4,436,658, or produced recombinantly as discussed in U.S. Pat. Nos. 6,100,054; 6,080,559; 6,066,469; 5,955,316; 5,849,881; 5,766,939; 5,571,896; 5,571,697; 5,571,691. Synthetic sources of lactoferrin can also be used where available. In some preferred embodiments, the lactoferrin, variant or fragment thereof is capable of binding to the leukocyte surface. The present methods also provide a technique, such as measuring the presence or absence of leukocyte binding, for assaying lactoferrin, derivatives and fragments with specific properties. When lactoferrin, variant or fragment thereof displays leukocyte binding properties, these proteins can further be assayed for the presence of additional properties disclosed herein, such as causing leukocyte activation and/or aggregation. The potency of the lactoferrin properties may also be measured based on the reactions.

It has been surprisingly and unexpectedly discovered that immobilized lactoferrin is capable of specifically binding to receptors on eosinophils and stimulating eosinophil activity, including stimulating superoxide production, leukotriene production, and degranulation. Many of the effects of lactoferrin on immune and inflammatory cell function that have been described to date are inhibitory in nature, including the inhibition of several LPS-stimulated responses (Vorland, L. H., Apmis 107:97 1 (1999); Baveye, S., E., et al., Clin. Chem. Lab. Med. 37:281 (1999)). In contrast, the present invention demonstrates that lactoferrin, specifically immobilized lactoferrin in concentrations similar to those present in airway surface liquid (Travis, S. M., et al., Am. J. Respir. Cell. Mol. Biol. 20:872 (1999)) is an effective stimulus for eosinophil superoxide production, degranulation and leukotriene C4 production.

The specificity of which immobilized lactoferrin binds and participates in the activation of eosinophils provides a method for assaying a wide array of compounds that regulate the activation of eosinophils. Compounds that interfere with the interaction between lactoferrin and eosinophils have implications for a number of disorders and conditions, including treatment of eosinophilia disorders and asthma. Other implications exist as compounds which stimulate eosinophil activation can be useful for enhancing weakened immune responses.

Accordingly, in an embodiment of the present invention, lactoferrin, derivatives and fragments thereof can be used as part of an assay to measure the ability of a test agent, such as a potential regulatory factor, compound or agent to induce cellular activity in eosinophils or other leukocytes. The potential regulatory factors can have inhibitory or stimulatory activity. According to these methods, leukocytes are contacted with lactoferrin in the presence of the potential regulatory factor and the level of activation of the leukocyte is determined or measured. The leukocyte activation can then be compared to a control reaction, if desired, where the leukocytes are contacted with the lactoferrin under the same conditions except that the potential regulator of leukocyte activation is excluded from the assay. Accordingly, the present methods are useful for identifying and testing drug candidates that help modulate leukocyte-mediated immune reactions. In some of these embodiments, potential regulators of lactoferrin-mediated leukocyte activation can be tested in the presence or absence of other, sometimes known, regulators of such activation such as GM-CSF.

After a test agent is identified as having a desired property, such as inhibiting, preventing or stimulating lactoferrin-mediated leukocyte activation, the test agent can be identified and then either isolated or chemically synthesized to produce a therapeutic drug. Thus, the present methods can be used to make drug products useful for the therapeutic treatment of lactoferrin-mediated leukocyte activation in vitro or in vivo. Agents identified as having a desired property can further be tested for specific activities, such as preventing leukocyte activation and/or aggregation. Accordingly, identified agents can have indications for preventing and treating allergic diseases such as bronchial asthma, dermatitis, rhinitis and conjunctivitis; autoimmune diseases such as rheumatoid arthritis, nephritis, Sjogren's syndrome, inflammatory bowel diseases, diabetes and arteriosclerosis; and chronic inflammatory diseases.

The present invention also provides kits for carrying out the methods described herein. In one embodiment, the kit is made up of instructions for carrying out any of the methods described herein. The instructions can be provided in any intelligible form through a tangible medium, such as printed on paper, computer readable media, or the like. The present kits can also include one or more reagents, buffers, media, proteins, such as lactoferrin or GM-CSF, analytes, labels, computer programs for analyzing results and/or disposable lab equipment, such as culture dishes or multi-well plates, in order to readily facilitate implementation of the present methods. Solid supports can include beads, culture dishes, multi-well plates and the like. Examples of preferred kit components can be found in the description above and in the following examples.

The present methods are further illustrated by the following non-limiting examples.

### EXAMPLES

The following materials and methods apply to the Examples that follow, unless otherwise indicated.

Cell Isolation

Neutrophils were isolated from venous blood of healthy adult volunteers by density gradient centrifugation through lymphocyte separation medium (BioWhittaker; Walkersville, Md.) as described previously (Haskell M. D., et al., Blood 86:4627-4637 (1995)) with one modification. Isotonicity was restored following the brief hypotonic lysis steps by the addition of 2× concentrated Hank's Balanced Salt Solution (HBSS) (Gibco; Grand Island, N.Y.) (without Ca²⁺ and Mg²⁺) containing 5 mM HEPES, pH 7.4. The cells were suspended in HEPES (10 mM)-buffered HBSS (with Ca²⁺ and Mg²⁺), pH 7.4, containing 1 milligram per milliliter of human serum albumin (HSA) (Sigma Chemical Co.) (HEPES-HBSS-HSA buffer). Neutrophil purity was routinely greater than 95 percent, with eosinophils representing the remainder of the cells. Eosinophils were isolated from the neutrophil preparations by negative selection (Hansel T. T., et al., J. Immunol. Meth. 145:105-10 (1991)) using anti-CD16 immunomagnetic beads as described by the manufacturer (Miltenyi Biotec, Inc., Auburn, Calif.). The eosinophils were suspended in HEPES-HBSS-HSA buffer. Eosinophil purity was routinely greater than 95 percent as determined by counting Wright-stained cytospin preparations. In some assays, an aliquot of the neutrophil preparation was held on ice for later use.

Superoxide Production

Superoxide production was measured essentially as described elsewhere (Motegi, Y., and H. Kita, J. Immunol. 161:4340 (1998)). Briefly, wells in a 96-well (flat bottom) tissue culture plate (Corning, Inc.; Corning, N.Y.) were coated with human milk lactoferrin (Sigma Chemical Co.) or human secretory IgA (ICN Biomedical; Aurora, Ohio) by incubation with 50 microliters of the indicated concentrations of the proteins in phosphate buffered saline (PBS) overnight at 4° C. Non-specific protein binding sites were blocked by subsequent incubation with 100 microliters of 25 milligrams per milliliter of HSA in PBS for 2 hours at 37° C., and the tissue culture wells were washed twice with PBS before use. Aliquots (5×10⁴ cells) of eosinophils or neutrophils were added to the wells and were incubated in HEPES-HBSS—HSA buffer containing 50 micromolar cytochrome c (Sigma Chemical Co.) for 120 minutes at 37° C. in a Ceres UV900HDi microplate reader (Bio-Tek Instruments, Inc., Winooski, Vt.). Total incubation volume was 0.2 milliliters. Absorbance at 550 nanometers was recorded at 15-minute intervals, and superoxide production was calculated as described previously (Motegi, Y., and H. Kita, J. Immunol. 161:4340 (1998)). Results are expressed as nanomoles superoxide per 10⁵ cells after subtraction of spontaneous production, which was measured in tissue culture wells coated only with HSA. GM-CSF (R & D Systems; Minneapolis, Minn.), porcine heparin (Sigma Chemical Co.), or chondroitin sulfate C (Sigma Chemical Co.) was added to the incubation mixtures in some assays as indicated.

In the examples, all statistical analyses were performed using Student's paired t-test. Statistical significance was set at p<0.05.

### EXAMPLE 1

### Immobilized Lactoferrin Stimulation of Eosinophils

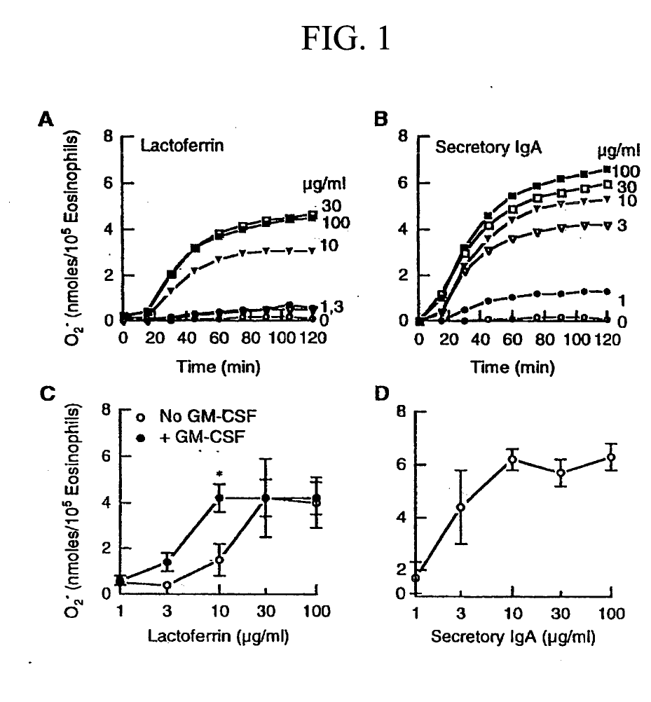

The capacity of immobilized lactoferrin to stimulate eosinophil superoxide production was examined by incubating eosinophils in tissue culture wells preincubated with 1 to 100 micrograms per milliliter of lactoferrin overnight at 4° C. This concentration range corresponds to the concentrations of lactoferrin measured in airway surface liquid (Travis, S. M., et al., Am. J. Respir. Cell. Mol. Biol. 20:872 (1999)) and to the effective concentration range for stimulation of eosinophil superoxide production by immobilized secretory IgA (Motegi, Y., and H. Kita, J. Immunol. 161:4340 (1998)). The results presented in FIG. 1A show that lactoferrin immobilized at concentrations of 10 micrograms per milliliter or greater stimulated marked superoxide production over the 2-hour incubation period. After an approximately 15-minute lag, superoxide production increased with time over the subsequent 45 to 60 minutes of incubation and then reached a plateau. Superoxide production stimulated by immobilized secretory IgA displayed a similar time course in the same assay (FIG. 1B).

Plotting the level of superoxide production measured at the 2-hour time point in five assays as a function of the concentration of immobilized lactoferrin or immobilized secretory IgA confirmed the concentration effects illustrated in FIGS. 1A and 1B. Specifically, immobilized lactoferrin at concentrations less than 3 micrograms per milliliter did not appear to stimulate superoxide production, whereas 30 micrograms per milliliter of immobilized lactoferrin appeared to produce a maximum response of approximately 5 nanomoles superoxide per 10⁵ eosinophils (FIG. 1C). The addition of 100 picograms per milliliter of GM-CSF to the incubation mixture enhanced the amount of superoxide production stimulated by 10 micrograms per milliliter of immobilized lactoferrin (FIG. 1C), but GM-CSF did not appear to increase the level of superoxide production stimulated by the higher concentrations of immobilized lactoferrin. Immobilized secretory IgA stimulated a concentration-dependent superoxide production over the same concentration range (FIG. 1D), as reported previously (Motegi, Y., and H. Kita, J. Immunol. 161:4340 (1998)). The amount of superoxide production stimulated by immobilized secretory IgA in these assays was about 50 percent greater than that stimulated by immobilized lactoferrin and peaked at the 10 microgram per milliliter concentration (in the absence of GM-CSF). In other results (n=4), incubating eosinophils with suboptimal concentrations (3 or 10 micrograms per milliliter) of immobilized lactoferrin and suboptimal concentrations (1 or 3 micrograms per milliliter) of immobilized secretory IgA in combination resulted in additive levels of superoxide production.

### EXAMPLE 2

### Immobilized Transferrin Stimulation of Eosinophils

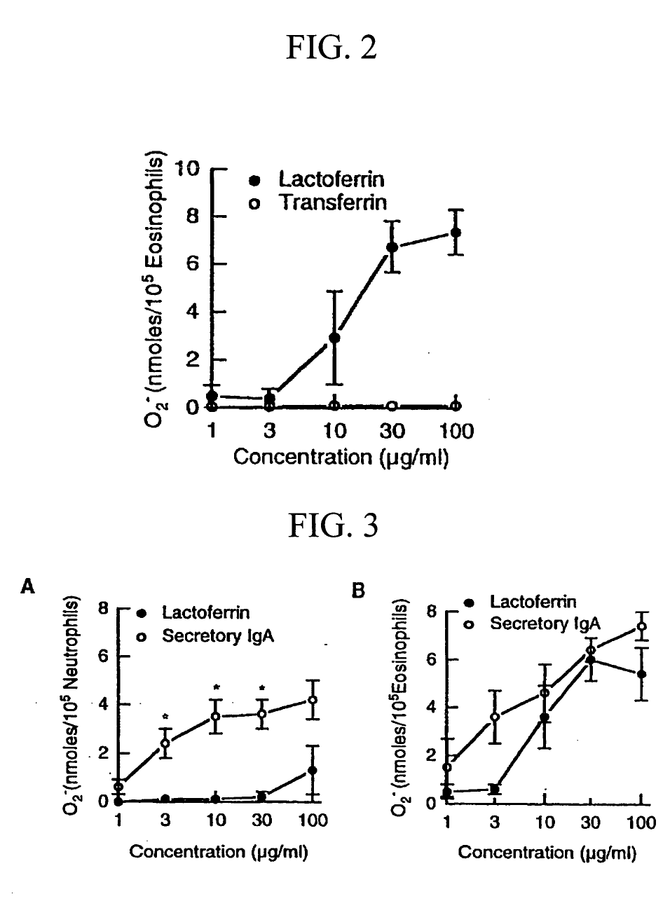

Because lactoferrin is a member of the transferrin family of proteins (Metz-Boutigue, M; H., et al., Eur. J. Biochem. 145:659 (1984)), the possibility that immobilized transferrin might also stimulate eosinophil superoxide production was evaluated. Incubating eosinophils with 1 to 100 micrograms per milliliter of immobilized transferrin did not stimulate superoxide production (FIG. 2). In contrast, when used in the same assays described above with lactoferrin, immobilized lactoferrin stimulated superoxide production by the eosinophils.

### EXAMPLE 3

### Immobilized Lactoferrin Stimulation of Neutrophils

The capacity of immobilized lactoferrin to stimulate superoxide production was examined using neutrophils and eosinophils isolated from the same donors. The results presented in FIG. 3A show that incubating neutrophils with 1 to 100 micrograms per milliliter of immobilized lactoferrin for up to 2 hours did not produce significant superoxide production. In contrast, the same concentrations of immobilized secretory IgA stimulated marked superoxide production by the neutrophils, with a time course and concentration-dependence similar to that observed for eosinophil superoxide production in the same assays (FIG. 3B). Only at the 100 microgram per milliliter concentration did the level of superoxide production stimulated by immobilized lactoferrin not differ significantly from that stimulated by immobilized secretory IgA. The amount of neutrophil superoxide production stimulated by 100 microgram per milliliter immobilized lactoferrin, however, was only 25 percent of the level of eosinophil superoxide production (5.5±1.4 nanomoles per 10⁵ eosinophils) stimulated by 30 micrograms per milliliter of immobilized lactoferrin in the same assays (FIG. 3B). In these assays immobilized lactoferrin and immobilized secretory IgA stimulated similar levels of superoxide production by the eosinophils (FIG. 3B).

### EXAMPLE 4

### Lactoferrin Binding to Eosinophils

Flow Cytometry

Eosinophils (10⁶ cells) were incubated with or without the indicated concentrations of lactoferrin in 100 microliters of HEPES-HBSS-HSA buffer for 90 minutes at 4° C. The cells were collected by centrifugation at 300 g for 5 minutes at 4° C., and then incubated with 1.5 micrograms FITC-conjugated polyclonal anti-lactoferrin (Sigma Chemical Co.) or FITC-conjugated rabbit IgG (Sigma Chemical Co.) in 25 microliters PBS (pH 7.2) containing 0.1 percent gelatin and 0.1 percent azide (PBS-gel-azide) for 30 minutes on ice. The cells were washed twice in ice-cold PBS-gel-azide and were suspended in PBS-gel-azide buffer containing 1 percent formaldehyde for analysis by flow cytometry. Fluorescence intensity of 10,000 cells in each sample was measured using a FACScan flow cytometer (Becton-Dickinson; San Jose, Calif.).

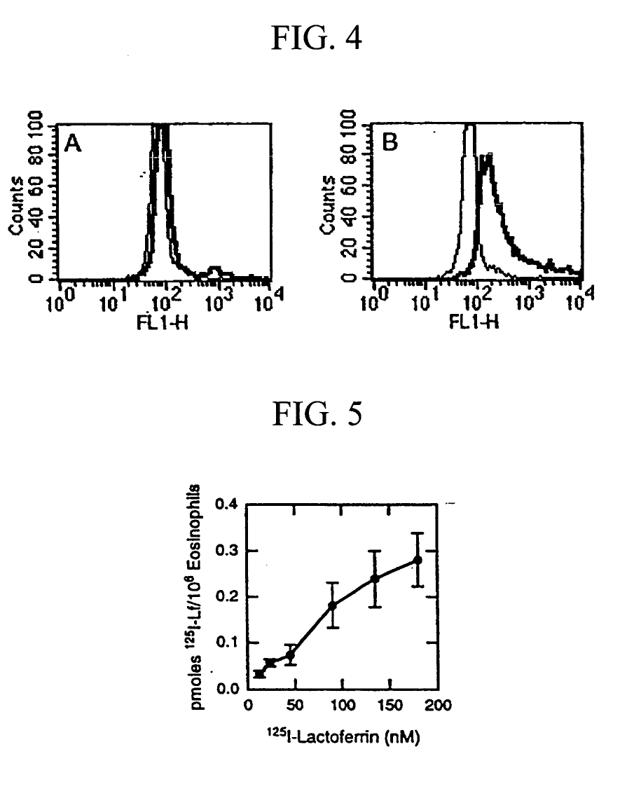

To confirm that eosinophils bind lactoferrin, eosinophils were incubated with or without 30 micrograms per milliliter of soluble lactoferrin for 90 minutes at 4° C. The presence of bound lactoferrin then was determined by flow cytometry using FITC-conjugated IgG anti-human lactoferrin or FITC-conjugated normal rabbit IgG. In the absence of lactoferrin, the FITC-conjugated anti-lactoferrin antibody did not display any specific reactivity with eosinophils (FIG. 4A). In contrast, incubating the eosinophils with 30 micrograms per milliliter of lactoferrin produced a marked increase in the fluorescence intensity following reaction with the FITC-conjugated anti-lactoferrin antibody (FIG. 4B). Incubating eosinophils with 100 micrograms per milliliter of lactoferrin did not produce any further increase in the level of fluorescence intensity with the FITC-anti-lactoferrin antibody. Similar results were obtained in two additional assays.

Binding experiments using labeled lactoferrin were performed to examine further the binding of lactoferrin by eosinophils.

Binding of Radiolabeled Lactoferrin

Lactoferrin (100 micrograms) was radioiodinated using IODO-GEN iodination reagent (Pierce; Rockford, Ill.) according to the procedure supplied by the manufacturer. The lactoferrin was incubated with 400 micro Curies Na¹²⁵I (PerkinElmer Life Sciences; Boston, Mass.) for 3 min, and the ¹²⁵I-labeled lactoferrin was separated from free Na¹²⁵I by chromatography through a 5-ml D-Salt dextran desalting column (Pierce) using PBS as the elution buffer. Protein concentration of the ¹²⁵I-labeled lactoferrin was measured by the BCA assay (Pierce). Specific activity of the ¹²⁵I-labeled lactoferrin was 34,800 cpm/pmole. Immobilized ¹²⁵I-labeled lactoferrin (30 micrograms per milliliter) retained full ability to stimulate eosinophil superoxide production (data not shown). Binding of ¹²⁵I-labeled lactoferrin by eosinophils was determined using a modification of previously described protocols for eosinophils. Motegi et al. J. Immunol. 161:4340 (1998); Lopez et al., J. Biol. Chem. 266:24741 (1991). Eosinophils (2×10⁶) were incubated with the indicated concentrations of ¹²⁵I-labeled lactoferrin alone and in the presence of excess unlabeled lactoferrin in RPMI 1640 containing 20 mM HEPES, 0.5% BSA, and 0.1% sodium azide (Lopez et al., J. Biol. Chem. 266:24741 (1991)) in siliconized glass tubes for 2 hr at room temperature on a circular oscillating platform. In some experiments as indicated, binding was measured in the presence of excess unlabeled transferrin. Total reaction volume was 0.15 ml. Reactions were stopped by centrifugation (1300 g for 4 min) of the reaction mixture through 200 microliters FCS in a 1.5 ml microcentrifuge tube. The supernatant was removed by careful aspiration, and after quick-freezing on dry ice the tip of the tube containing the cell pellet was excised and the radioactivity was measured by gamma counting (Beckman Coulter Gamma 5500B). Specific binding was determined as the difference between total binding and binding in the presence of the excess unlabeled lactoferrin. Binding constants were determined by Scatchard analysis (Scatchard, G., Ann. N.Y. Acad. Sci. 51:660 (1949)).

Incubating eosinophils with 23 nanomolar to 180 nanomolar ¹²⁵I-labeled lactoferrin alone and in the presence of 5 micromolar unlabeled lactoferrin for 2 hours at room temperature confirmed specific binding of the ¹²⁵I-labeled lactoferrin (FIG. 5). Binding approached saturation and suggested the presence of two binding affinities, suggesting two classes of receptors. Analysis of binding data obtained in three experiments demonstrated the presence of two classes of receptors: one with a K_{D} of 47+/−19 nanomolar (mean+/−SE) with a population of approximately 78,000+/−13,000 per eosinophil; and a second with a K_{D} Of approximately 260 nanomolar with a population of up to approximately 620,000 molecules per eosinophil. Eosinophil binding of ¹²⁵I-labeled lactoferrin at 90 nanomolar concentration was not inhibited in the presence of 5 micromolar transferrin (n=2).

These results confirm that soluble lactoferrin binds to eosinophils, as determined by flow cytometry. Lactoferrin receptors have been described previously for a variety of cells, including various leukocytes (Boxer, L. A., et al., J. Clin. Invest. 70:1049 (1982); Birgens, H. S., et al., Br. J. Haematol. 54:383 (1983); Miyazawa, K., et al., J. Immunol. 146:723 (1991); Ismail, M., and J. H. Brock, J. Biol. Chem. 268:216 18 (1993); Bi, B. Y., et al., Eur. J. Cell Biol. 69:288 (1996); Mincheva-Nilsson, L., et al., Scand. J. Immunol. 46:609 (1997)) and epithelial cells (Ghio, A. J., et al., Am. J. Physiol. 276:L933 (1999)). The binding affinities reported for the different cells vary widely, with the dissociation constants ranging from nanomolar to micromolar concentrations (Boxer, L. A., et al., J. Clin. Invest. 70:1049 (1982); Birgens, H. S., et al., Br. J. Haematol. 54:383 (1983); Ismail, M., and J. H. Brock, J. Biol. Chem. 268:21618 (1993); Bi, B. Y., et al., Eur. J. Cell Biol. 69:288 (1996); Mincheva-Nilsson, L., et al., Scand. J. Immunol. 46:609 (1997); Ghio, A. J., et al., Am. J. Physiol. 276:L933 (1999)). The results presented for binding of ¹²⁵I-lactoferrin by eosinophils suggest that eosinophils possess two classes of lactoferrin receptors, with dissociation constants of approximately 47 nM and 260 nM. Two classes of lactoferrin receptors have also been reported for the human promonocyte THP1 cell line (Roseanu, A., et al., Biochim. Biophys. Acta 1475:35 (2000)). It is likely that the apparent two classes of lactoferrin receptors reflect at least in part the relative structural complexity of the 78 kDa lactoferrin molecule (Spik et al., Adv. Exp. Med. Biol. 357:21 (1994); Mann et al., J. Biol. Chem. 269:23661 (1994); and Wu et al., Arch. Biochem. Biophys. 317:85 (1995)). The apparent number of lactoferrin receptor molecules expressed by eosinophils is less than that reported for other cells (Boxer, L. A., et al., J. Clin. Invest. 70:1049 (1982); Birgens, H. S., et al., Br. J. Haematol. 54:383 (1983); Ismail, M., and J. H. Brock, J. Biol. Chem. 268:21618 (1993); Bi, B. Y., et al., Eur. J. Cell Biol. 69:288 (1996); Mincheva-Nilsson, L., et al., Scand. J. Immunol. 46:609 (1997); and Roseanu, A., et al., Biochim. Biophys. Acta 1475:35 (2000)). Although complete saturation was not achieved in the binding experiments using ¹²⁵I-lactoferrin, the results of the flow cytometry analysis suggest that binding of lactoferrin by eosinophils is saturated following incubation with 30 micrograms per milliliter of lactoferrin (approximately 0.4 micromolar lactoferrin). As demonstrated below in Example 5, soluble lactoferrin does not significantly activate eosinophils. The results that soluble lactoferrin does not significantly activate eosinophils, as measured by superoxide production, and does not completely block immobilized lactoferrin in concentrations up to 100 micrograms per milliliter (approximately 1 micromolar) suggest that lactoferrin-induced activation of eosinophils requires activation of a relatively low-affinity lactoferrin receptor. Although a lactoferrin receptor with a dissociation constant of approximately 200 nM has been described for neutrophils (Boxer, L. A., et al., J. Clin. Invest. 70:1049 (1982)) immobilized lactoferrin does not stimulate neutrophil superoxide production.

The finding that soluble lactoferrin in concentrations up to 100 micrograms per milliliter (approximately 1 micromolar) did not block eosinophil activation by immobilized lactoferrin is consistent with a low-affinity binding site. The possible existence of a high-affinity lactoferrin receptor on eosinophils, however, cannot be excluded, as the binding of lower concentrations of lactoferrin by the eosinophils was not examined. It is of interest in this context that the lactoferrin receptor described on neutrophils has a dissociation constant of approximately 0.2 micromolar and is saturated by incubation with 100 to 200 nanomolar lactoferrin (Boxer, L. A., et al., J. Clin. Invest. 70:1049 (1982)). Still, the results here show that neutrophils are not responsive to immobilized lactoferrin, at least as determined by superoxide production.

### EXAMPLE 5

### Soluble Lactoferrin Stimulation of Eosinophils

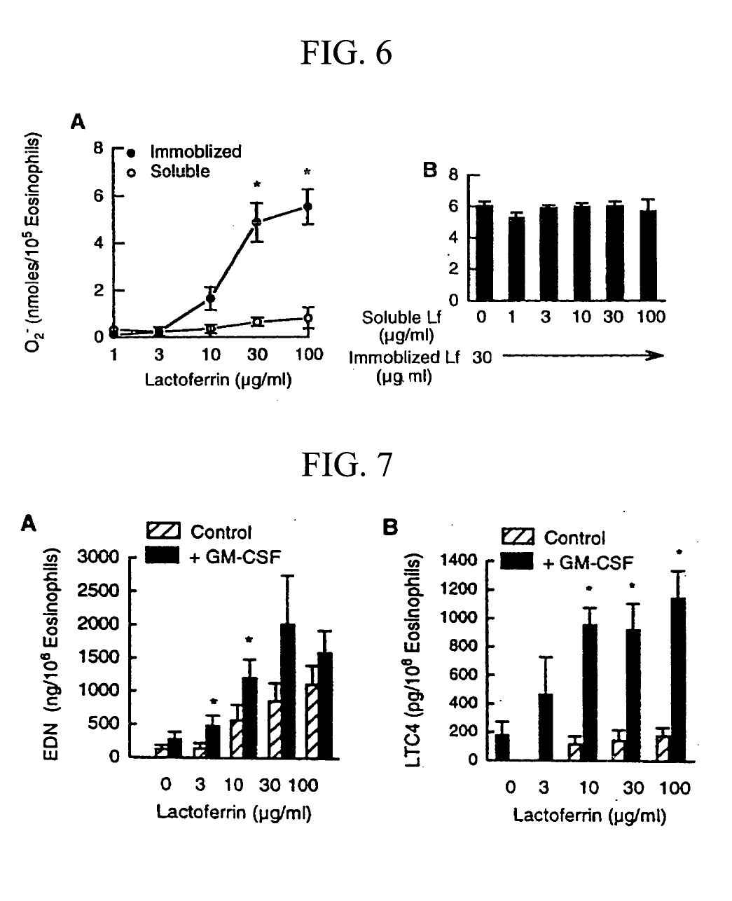

The capacity of soluble lactoferrin to stimulate eosinophil superoxide production was examined by incubating eosinophils with 1 to 100 micrograms per milliliter of soluble lactoferrin for 2 hours at 37° C. in tissue culture wells coated only with HSA. The results presented in FIG. 6A show that soluble lactoferrin stimulated minimal superoxide production by the eosinophils, whereas immobilized lactoferrin stimulated superoxide production in the expected concentration-dependent manner in the same assays. Additional assays demonstrated that addition of 1 to 100 micrograms per milliliter of soluble lactoferrin did not inhibit eosinophil superoxide production stimulated by 30 micrograms per milliliter of immobilized lactoferrin (FIG. 6B).

### EXAMPLE 6

### Immobilized Lactoferrin in Eosinophil Degranulation and Leukotriene C4 Release

The capacity of immobilized lactoferrin to stimulate eosinophil degranulation was assessed by EDN release after incubating eosinophils with 3 to 100 micrograms per milliliter of immobilized lactoferrin for 4 hours at 37° C. in a 5 percent CO₂ atmosphere.

Degranulation

Eosinophils (2×10⁵) were incubated in the presence and absence of 100 picograms per milliliter of GM-CSF in RPMI 1640 containing 1 milligram per milliliter of HSA for 4 hours at 37° C. in 5 percent CO₂ in tissue culture wells precoated with lactoferrin or secretory IgA as above. Total incubation volume was 0.2 milliliters. Reactions were stopped by centrifugation at 300 g for 5 minutes at 4° C., and supernatants were stored at −20° C. until measurement of eosinophil-derived neurotoxin (EDN) content by specific ELISA (MBL International, Watertown, Mass.). Spontaneous release of EDN was determined with cells incubated in HSA-coated wells.

The results presented in FIG. 7A show that immobilized lactoferrin stimulated the net release of up to approximately 1000 nanograms of EDN per 10⁶ eosinophils in a concentration-dependent manner, with maximum release observed at the 100 micrograms per milliliter concentration. The addition of 100 picograms per milliliter of GM-CSF significantly enhanced EDN release stimulated by 3 and 10 micrograms per milliliter of immobilized lactoferrin. In the same assays, immobilized secretory IgA stimulated the net release of approximately 500 nanograms per milliliter of EDN at each of the concentrations tested over the range of 3 to 100 micrograms per milliliter.

Leukotriene C4 Production

Eosinophils (2×10⁵) were incubated in the presence and absence of 100 pg/ml GM-CSF in RPMI 1640 containing 10 mM HEPES for 1 hr at 37° C. in tissue culture wells precoated with lactoferrin or secretory IgA as above with one modification. The tissue culture wells were not treated with HSA and HSA was not added to the incubation buffer to minimize loss of leukotriene C4. Total incubation volume was 0.2 ml. Reactions were stopped by centrifugation at 300 g for 5 min at 4° C., and supernatants were stored at −20° C. until measurement of leukotriene C4 content by a leukotriene C4/D4/E4 ELISA (Amersham Pharmacia Biotech; Piscataway, N.J.). Spontaneous leukotriene C4 production was determined with eosinophils incubated in untreated tissue culture wells.

The effect of immobilized lactoferrin on leukotriene C4 production by eosinophils was evaluated in additional experiments. Incubating eosinophils with 3 to 100 micrograms per milliliter immobilized lactoferrin stimulated only low levels of leukotriene C4 over a 1 hour incubation period (FIG. 7B). The addition of 100 picograms per milliliter GM-CSF, however, significantly enhanced leukotriene C4 release stimulated by 10 to 100 micrograms per milliliter immobilized lactoferrin. Whereas incubation with 10 micrograms per milliliter immobilized lactoferrin alone and 100 picograms per milliliter GM-CSF alone stimulated the production of 115 and 175 picograms leukotriene C4 per 10⁶ eosinophils, respectively, incubation with the two stimuli together resulted in production of 955 picograms leukotriene C4 per 10⁶ eosinophils.

### EXAMPLE 7

### Eosinophil Activation by Immobilized Deglycosylated Lactoferrin

To assess whether the N-linked oligosaccharides in lactoferrin (Spik, G., et al., Adv. Exp. Med. Biol. 357:21 (1994)) contributed to eosinophil activation, the activities of lactoferrin deglycosylated by PNGase F treatment and lactoferrin treated in the same manner but in the absence of PNGase F (mock-deglycosylated lactoferrin) were compared.

Deglycosylated Lactoferrin

Lactoferrin and transferrin differ slightly in the composition of their N-linked oligosaccharides, specifically in the presence of a fucose (a-1,6) residue in the core of the lactoferrin N-linked oligosaccharides (Spik, G., et al., Adv. Exp. Med. Biol. 357:21 (1994)). Similar to the findings here, the high affinity binding of lactoferrin to the human pro-monocytic U937 cell line also appears to occur independently of fucosyl or glycosyl residues and is not blocked by heparinase treatment of the cells (Ismail, M., and J. H. Brock, J. Biol. Chem. 268:21618 (1993)). Also similar to the findings here, transferrin does not inhibit the binding of lactoferrin by HL-60 cells before or after induced differentiation toward monocyte/macrophage-like cells, by human monocytes, or by the U937 cells (Birgens, H. S., et al., Br. J. Haematol. 54:3 83 (1983); Miyazawa, K., et al., J. Immunol. 146:723 (1991); Ismail, M., and J. H. Brock, J. Biol. Chem. 268:21618 (1993)).

Lactoferrin (1 milligram per milliliter) was incubated without (mock-deglycosylated) or with 105 units per milliliter of peptide N-glycosidase F (PNGase F) (New England Biolabs; Beverly, Mass.) in PBS for 72 hours at 37° C. Following incubation, the lactoferrin was stored in aliquots at −70° C. until use. Deglycosylation was assessed by a reduction in the apparent Mᵣ as determined in Coomassie Blue-stained SDS-PAGE gels and by reactivity with HRP-conjugated Con A (E-Y Laboratories; San Mateo, Calif.). For Coomassie-stained gels, 10 micrograms of protein was subjected to SDS-PAGE in 8 percent gels under non-reducing conditions (Laemmli, U. K., Nature 227:680 (1970)). For reactivity with HRP-conjugated Con A, 0.2 micrograms of protein was subjected to SDS-PAGE as above and was transferred electrophoretically to Hybond ECL nitrocellulose (Amersham Pharmacia Biotech; Piscataway, N.J.). After blocking with 3 percent gelatin in TBST, the membrane was incubated with 0.2 micrograms per milliliter of HRP-conjugated Con A in TBST containing 3 percent gelatin for 1 hour at room temperature. The blot was washed extensively with TBST, and positive bands were visualized by ECL.

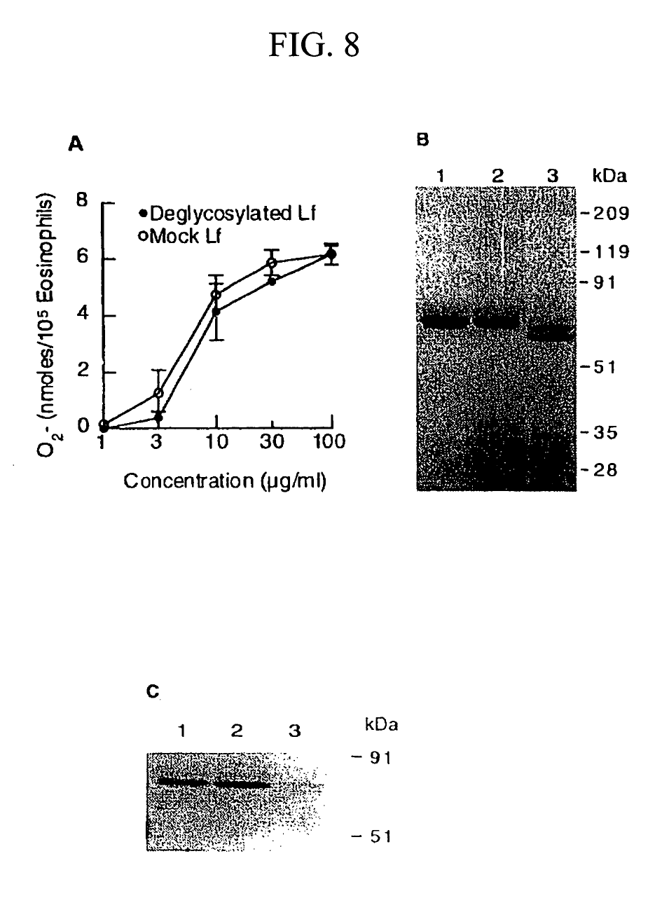

The results show that incubating eosinophils with 1 to 100 micrograms per milliliter of immobilized deglycosylated lactoferrin stimulated superoxide production to the same extent and in the same concentration-dependent manner as immobilized mock-deglycosylated lactoferrin (FIG. 8A). In the same assay, the effect of mock-deglycosylated lactoferrin did not differ from that of control lactoferrin. Deglycosylation of the PNGase F-treated lactoferrin was confirmed by reduction in the Mᵣ of the protein in SDS-PAGE (FIG. 8B) and by diminished reactivity with Con A (FIG. 8C).

### EXAMPLE 8

### Effect of Heparin and Chondroitin Sulfate on Lactoferrin-Mediated Eosinophil Activation

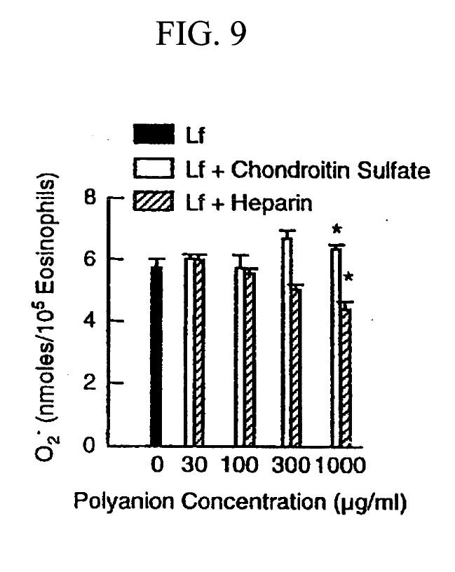

The effects of heparin and chondroitin sulfate on eosinophil activation by immobilized lactoferrin were evaluated in three additional assays to assess the involvement of the putative glycosaminoglycan-binding site in lactoferrin (Mann, D. M., et al., J. Biol. Chem. 269:2366 1 (1994); Wu, H. F., et al., Arch. Biochem. Biophys. 3 17:85 (1995)) in the response. The addition of 30 to 1000 micrograms per milliliter of heparin inhibited superoxide production stimulated by 30 micrograms per milliliter of immobilized lactoferrin by approximately 25 percent at the highest concentration tested (FIG. 9). Heparin had a similar effect on superoxide production stimulated by 10 micrograms per milliliter immobilized secretory IgA in the same experiments, with 1000 micrograms per milliliter heparin inhibiting the response by 28+/−11 percent. The inhibition did not achieve statistical significance (p=0.07). Chondroitin sulfate at 1000 micrograms per milliliter caused a slight, but statistically significant, increase in superoxide production stimulated by the immobilized lactoferrin.

The results presented here for heparin and chondroitin sulfate indicate that the glycosaminoglycan-binding site likely does not play a role in the eosinophil activation by immobilized lactoferrin. Heparin at a concentration of 1 milligram per milliliter caused only modest inhibition (25 percent) of eosinophil superoxide production stimulated by 30 micrograms per milliliter of immobilized lactoferrin. In contrast, neither the lower concentrations of heparin nor any of the concentrations of chondroitin sulfate inhibited the eosinophil superoxide production.

### DISCUSSION OF EXAMPLES

Incubating eosinophils in tissue culture wells pretreated with 1 to 100 micrograms per milliliter lactoferrin (isolated from human milk) stimulated concentration-dependent superoxide production by eosinophils. Immobilized lactoferrin also stimulated the release of eosinophil-derived neurotoxin (EDN). In contrast, the same concentrations of immobilized transferrin had no effect on superoxide production. The potency of the immobilized lactoferrin was approximately one-third the potency of immobilized secretory IgA in the same assays. As shown in Example 3, immobilized lactoferrin was not as efficient as stimulating neutrophil superoxide production compared to secretory IgA. Eosinophils bound lactoferrin as determined by reactivity with FITC-anti-human lactoferrin after incubating the eosinophils with 30 micrograms per milliliter of soluble lactoferrin for 90 minutes at 40° C. and by bindinh ¹²⁵I-labeled lactoferrin. Transferrin did not block binding of ¹²⁵I-labeled lactoferrin. Interestingly, soluble lactoferrin did not activate the eosinophils as well as immobilized lactoferrin and did not appear to block superoxide production stimulated by immobilized lactoferrin. Immobilized lactoferrin also stimulated release of eosinophil-derived neurotoxin and resulted in low levels of leukotriene C4 production. The presence of 100 picograms per milliliter of Granulocyte Macrophage Colony Stimulating Factor (GM-CSF) enhanced superoxide production and EDN release in conjunction with lower concentrations of immobilized lactoferrin. Pretreatment of the lactoferrin with peptide N-glycosidase F or the presence of chondroitin sulfate had no or minimal effect on the activity of immobilized lactoferrin. At higher concentrations, the presence of heparin had an inhibitory effect on the activity of immobilized lactorferrin. These results demonstrate that lactoferrin adherent to the surface epithelium of the lungs may contribute to the activation of eosinophils that infiltrate the airway lumen in eosinophil-associated disorders such as asthma.

Eosinophil activation was triggered by lactoferrin that had been immobilized at concentrations greater than 3 micrograms per milliliter, and the maximum or near-maximum response appeared to exist in tissue culture wells that had been preincubated with 30 micrograms per milliliter of lactoferrin. Similar to the effect of GM-CSF on superoxide production and degranulation stimulated by other eosinophil stimuli (Nagata, M., et al., J. Immunol. 155:4948 (1995); Horie, S., et al., J. Allergy Clin. Immunol. 98:371 (1996); Fujisawa, T., et al., J. Immunol. 144:642 (1990)), the presence of a low concentration (100 picograms per milliliter) of GM-CSF significantly enhanced the level of eosinophil superoxide production and EDN release stimulated by immobilized lactoferrin. GM-CSF, however, enhanced the eosinophil responses only when the cells were stimulated by the lower concentrations of immobilized lactoferrin. The net result of the GM-CSF presence, thus, appears to reduce the concentration of immobilized lactoferrin required to stimulate the maximal superoxide production or EDN release by approximately three-fold, although this effect was most evident for superoxide production (FIG. 1C). GM-CSF also markedly enhanced eosinophil leukotriene C4 production stimulated by concentrations of immobilized lactoferrin greater than 3 micrograms per milliliter. In the absence of GM-CSF, immobilized lactoferrin stimulated levels of leukotriene C4 release less leukotriene C4 than those reported previously for immobilized IgG (Moqbel, R., et al., Immunology 69:435 (1990); Bartemes, K. R., et al., J. Immunol. 162:2982 (1999)) but similar to levels reported for fMLP (Takafuji, S., et al., J. Immunol. 147:3855 (1991); Bates, M. E., et al., J. Biol. Chem 275:10968 (2000)). The level of leukotriene C4 production stimulated by the immobilized lactoferrin in the presence of GM-CSF is similar to that obtained with fMLP for IL-5 primed eosinophils (Takafuji, S., et al., J. Immunol. 147:3855 (1991); Bates, M. E., et al., J. Biol. Chem 275:10968 (2000)).

Immobilized lactoferrin, although appearing to be approximately one-third as potent as immobilized secretory IgA, is on occasion (FIG. 3B) nearly as efficacious as immobilized secretory IgA in stimulating eosinophil superoxide production and EDN release.

Although lactoferrin is a member of the transferrin family of proteins and shares 60 percent sequence identity with serum transferrin (Metz-Boutigue, M. H., et al., Eur. J. Biochem. 145:659 (1984)) immobilized transferrin did not stimulate eosinophil activation as measured by superoxide production. Thus, the activity of immobilized lactoferrin does not appear to be conserved among members of the transferrin family of proteins. Lactoferrin contains a glycosaminoglycan-binding site near its amino terminus (Mann, D. M., et al., J. Biol. Chem. 269:23661 (1994); Wu, H. F., et al., Arch. Biochem. Biophys. 317:85 (1995)) that is absent in transferrin (Metz-Boutigue, M. H., et al., Eur. J. Biochem. 145:659 (1984)). This site has been implicated in a low-affinity binding of lactoferrin by THP-1 cells (Roseanu, A., et al., Biochim. Biophys. Acta 1475:35 (2000)) and also mediates binding of LPS by lactoferrin (Baveye, S., et al., Clin. Chem. Lab. Med. 37:28 1 (1999)).

Interestingly, the responses stimulated by immobilized lactoferrin and immobilized secretory component, a polypeptide produced by cells of some secretory epithelia involved in transporting secreted polymeric IgA across the cell and protecting it from digestion in the gastrointestinal tract, share a trait in common. Specifically, immobilized lactoferrin and immobilized secretory component each stimulate eosinophil superoxide production but not neutrophil superoxide production (FIG. 3A; Motegi, Y., and H. Kita, J. Immunol. 161:4340 (1998)). Eosinophil activation by immobilized secretory component is correlated with the presence of a putative 15-kDa receptor for secretory component on eosinophils that is absent in neutrophils (Lamkhioued, B., et al., Eur. J. Immunol. 25:117 (1995)). Although these findings suggest that immobilized lactoferrin may cross-react with the putative receptor for secretory component on eosinophils (Lamkhioued, B., et al., Eur. J. Immunol. 25:117 (1995)), it is of interest that S. pneumoniae have specific and distinct receptors for lactoferrin and secretory component on their surface (Hammerschmidt, S., et al., Mol. Microbiol. 25:1113 (1997); Hammerschmidt, S., et al., Infect. Immun. 67:1683 (1999); Hakansson, A., et al., Infect. Immun. 69:3372 (2001)). The latter results at least raise the possibility that lactoferrin and secretory component may likewise recognize distinct receptors on eosinophils. Analogous to the results presented here, transferrin does not bind to the pneumococcal receptor for lactoferrin (Hakansson, A., et al., Infect. Immun. 69:3372 (2001)).

The present methods can involve any or all of the steps or conditions discussed above in various combinations, as desired. Accordingly, it will be readily apparent to the skilled artisan that in some of the disclosed methods certain steps can be deleted or additional steps performed without affecting the viability of the methods.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” “more than” and the like include the number recited and refer to ranges which can be subsequently broken down into subranges as discussed above. In the same manner, all ratios disclosed herein also include all subratios falling within the broader ratio.

One skilled in the art will also readily recognize that where members are grouped together in a common manner, such as in a Markush group, the present invention encompasses not only the entire group listed as a whole, but each member of the group individually and all possible subgroups of the main group. Accordingly, for all purposes, the present invention encompasses not only the main group, but also the main group absent one or more of the group members. The present invention also envisages the explicit exclusion of one or more of any of the group members in the claimed invention.

All references disclosed herein are specifically incorporated by reference thereto.

While preferred embodiments have been illustrated and described, it should be understood that changes and modifications can be made therein in accordance with ordinary skill in the art without departing from the invention in its broader aspects as defined in the following claims.

What is claimed is:

1. A method for assaying leukocyte activation, comprising:
(a) contacting one or more leukocytes with lactoferrin, a portion of lactoferrin or a derivative of lactoferrin or a portion thereof; and
(b) determining whether the one or more leukocytes are activated by the contact with the lactoferrin, the portion of lactoferrin or the derivative of lactoferrin or a portion thereof.

2. The method of claim 1 wherein the lactoferrin, the portion of lactoferrin or the derivative of lactoferrin or a portion thereof are immobilized on a surface.

3. The method of claim 2 wherein the surface comprises a multi-well plate, a polymer or metal bead, a cell layer or a culture dish.

4. The method of claim 1 wherein the leukocytes comprise one or more eosinophils, one or more neutrophils and combinations thereof.

5. The method of claim 4 wherein the leukocytes comprise about 95% purity of either the one or more eosinophils or the one or more neutrophils.

6. The method of claim 1 wherein step (b) comprises: (i) detecting superoxide production by the one or more leukocytes, (ii) detecting eosinophil-derived neurotoxin (EDN) release by the one or more leukocytes; (iii) detecting degranulation of the one or more leukocytes; (iv) detecting production of one or more leukotrienes; (v) detecting whether the lactoferrin, the portion of lactoferrin or derivative of lactoferrin or a portion thereof binds to the leukocyte; (vi) detecting production of one or more cytokines; and (vii) combinations of (i)-(vi).

7. The method of claim 6 wherein step (b) further comprises quantifying the level of activation of the one or more leukocytes.

8. The method of claim 1 wherein step (b) further comprises quantifying the level of activation of the one or more leukocytes.

9. The method of claim 1 further comprising (c) immobilizing the lactoferrin, the portion of lactoferrin or derivative of lactoferrin or a portion thereof on a surface.

10. The method of claim 1 wherein the concentration of the lactoferrin, the portion of lactoferrin or derivative of lactoferrin or a portion thereof is from about 1 to about 100 μg/ml.

11. The method of claim 1 wherein (a) is performed in the presence of one or more potential modulators of leukocyte activation.

12. The method of claim 11 further comprising: identifying one or more potential modulators of leukocyte activation that have desirable properties; and producing the one or more potential modulators of leukocyte activation as a therapeutic drug.

13. The method of claim 11 wherein the potential modulators of leukocyte activation are potential inhibitors or potential stimulators of leukocyte activation.

14. The method of claim 13 wherein the potential inhibitor of leukocyte activation decreases lactoferrin dependent leukocyte activation.

15. The method of claim 11 further comprising performing (a) in the absence of the one or more potential inhibitors or stimulators of leukocyte activation and comparing the leukocyte activation in the presence of the one or more potential inhibitors or stimulators of leukocyte activation with the leukocyte activation in the absence of the one or more potential inhibitors or stimulators of leukocyte activation.

16. The method of claim 11 further comprising performing (a) in the presence of one or more known modulators of leukocyte activation.

17. The method of claim 16 wherein the known modulator of leukocyte activation is granulocyte macrophage colony stimulating factor.

18. A method for assaying leukocyte activation, comprising:
(a) isolating a cell population consisting essentially of eosinophils from a patient;
(b) immobilizing lactoferrin, a portion of lactoferrin, a derivative of lactoferrin or a portion thereof on a solid surface to produce immobilized lactoferrin, a portion of lactoferrin, a derivative of lactoferrin or a portion thereof on a solid surface in a concentration of 15 micrograms per milliliter, wherein the immobilized lactoferrin, portion of lactoferrin, derivative of lactoferrin or portion thereof is capable of binding to an eosinophil;
(c) contacting a cell population consisting essentially of eosinophils with the immobilized lactoferrin, portion of lactoferrin, derivative of lactoferrin or portion thereof in the presence of 75 picograms per milliliter granulocyte macrophage colony stimulating factor; and
(d) determining whether the eosinophils in the cell population are activated by the contact with immobilized lactoferrin, portion of lactoferrin, derivative of lactoferrin or portion thereof by measuring an amount of produced superoxide.

19. A method for measuring the activity of an immobilized lactoferrin, portion of lactoferrin or derivative of lactoferrin or a portion thereof comprising:
(a) contacting immobilized lactoferrin, a portion of lactoferrin or a derivative of lactoferrin or a portion thereof with one or more eosinophils; and
(b) determining whether the immobilized lactoferrin, a portion of lactoferrin or a derivative of lactoferrin or a portion thereof activate the one or more eosinophils.

20. A kit for assaying leukocyte activation, comprising:
(a) instructions for carrying out any of the methods described herein; and
(b) one or more reagents for performing the described methods.
